(19) United States
(12) Plant Patent Application Publication
(10) Pub. No.: US 2003/0154531 P1
(43) Pub. Date: Aug. 14, 2003

(54) PETUNIA PLANT NAMED 'SUNROVE'

(76) Inventor: Kiyoshi Miyazaki, Hikone-shi (JP)

Correspondence Address: BURNS, DOANE, SWECKER & MATHIS, L.L.P., P.O. Box 1404, Alexandria, VA, 22313-1404, US

(21) Appl. No.: 10/067,802

(22) Filed: Feb. 8, 2002

(51) Int. Cl. A01H 5/00

(52) U.S. Cl. PLT/356

(57) ABSTRACT

Disclosed herein is a new and distinct variety of Petunia plant having a decumbent habit and long stems. The Petunia plant has abundant branching, and great profusion of blooms, the whole plant remaining in bloom for a considerable period of time. The flowers are single and medium size, the petals having a light purplish pink ground color with a vivid reddish purple vein pattern. The bottom color of the corolla throat is brilliant purple and the outside of the corolla tube is very pale violet. The plant exhibits high resistance to rain, heat and disease.

### BACKGROUND OF THE VARIETY

The present invention relates to a new and distinct variety of petunia plant originating from crossing a seedling called ‘6Pt-160-1’ (♀) and a wild type Petunia plant called ‘1792’ (♂).

The Petunia is a very popular plant that is used for flower bedding and potting in the summer season. There are only a few Petunia varieties which do not have an upright growth habit and which have a high resistance to rain, heat, and diseases. The Petunia plants that we previously filed, i.e. the ‘Revolution’ series {‘Revolution Purple pink’ (U.S. Plant Pat. No. 6,915), ‘Revolution Brilliant pink’ (U.S. Plant Pat. No. 6,914), ‘Revolution Brilliant pink-mini’ (U.S. Plant Pat. No. 6,899), ‘Revolution Bluevein’ (U.S. Plant Pat. No. 9,322), and ‘Revolution Pinkvein’ (U.S. Plant Pat. No. 9,341)} are decumbent type plants having long stems, a lower plant height, abundant branching, and a high resistance to heat, rain and disease. However, there are only a few Petunia varieties having a great profusion of flowers, pastel-colored with vein pattern flower petals and a high resistance to rain, heat, and diseases. Accordingly, this invention is aimed at obtaining a new variety having light purplish pink colored petals with a vivid reddish purple vein pattern, together with the above features.

The new variety of petunia plant according to this invention originated from crossing a seedling called ‘6Pt-160-1’ (♀) and a wild type Petunia plant called ‘1792’ (♂).

‘6Pt-160-1’ (not patented in the United States) was a seedling obtained from the ‘Falcon Plum’ (Sakata Seed Corp.) at Ohmi R&D Center, Flower Division, Suntory Ltd., located at 863-1, Aza-Iketani, Omori-cho, Yokaichi-shi, Shiga-ken, Japan. Variety ‘1792’ (not patented in the United States) is a decumbent, wild type Petunia plant native to Brazil. In April 1997, crossing ‘6Pt-160-1’ as female parent and the ‘1792’ as pollen parent was conducted at Ohmi R&D Center, Flower Division, Suntory Ltd. In January 1998, 120 seedlings were obtained from that crossing. These seedlings were grown in pots and tested and 2 seedlings were selected. One of them was selected in view of its decumbent growth habit and light purplish pink colored petals with a vivid reddish purple vein pattern. That seedling was propagated by cutting in July 1998, and a trial was carried out by flower potting and bedding from March 1999, at the Ohmi R&D Center, Flower Division, Suntory Ltd. The botanical characteristics of that plant were then examined, using a similar variety ‘Revolution Pinkvein’ for comparison. As a result, it was concluded that this petunia plant is distinguishable from other varieties whose existence is known, and uniform and stable in its characteristics. Then, the new variety of petunia plant was named ‘SUNROVE’.

In the following description, the color-coding is in accordance with the Horticultural Color Chart of The Royal Horticultural Society, London, England (R.H.S. Color Chart) and the Inter-Society Color Council-Nation Bureau of Standard Color Name (LS.C.C.-N.B.S. Color Name). A color chart based on The Japan Color Standard for Horticultural Plants (J.H.S. Color Chart) is also added for reference.

The female parent used in the crossing of ‘SUNROVE’ called ‘6Pt-160-1’ has an erect habit while that of the new variety is decumbent. The branching is less than the new variety. The petals are light purplish pink (R.H.S. 73C, J.H.S. 8903) with strong reddish purple (R.H.S. 72B, J.H.S. 9208) vein. The bottom color of the corolla throat is moderate reddish purple (R.H.S. 79C, J.H.S. 8913), and the outside color of the corolla tube is pale purple (R.H.S. 85D, J.H.S. 8914).

The main botanical characteristics of the ‘6Pt-160-1’ are as follows:

Plant:

Growth habit.—Erect.

Plant height.—18 cm.

Spreading area of plant.—Small.

Blooming period.—April to July in the southern Kanto area, Japan.

Stem:

Length.—13.5 cm.

Thickness.—Main stem 1.8 mm.

Pubescence.—Normal.

Branching.—Medium.

Length of internode.—1.8 cm.

Color.—Strong yellow green (R.H.S. 137C, J.H.S. 3512).

Leaf:

Whole shape.—Elliptic. The apex shape is acute, and the base shape is attenuate.

Length.—8.0 cm.

Width.—3.8 cm.

Color.—Upper-side color is moderate yellow green (R.H.S. 137C, J.H.S. 3712). Bottom-side color is moderate yellow green (R.H.S. 147B, J.H.S. 3513).

Pubescence.—Normal.

Flower:

Facing direction.—Upward.

Type.—Single.

Shape.—Funnel-shape, with five-fissured limb.

Shape of petal chip.—Round.

Lobation.—Shallow.

Waving of petal.—Weak.

Diameter.—5.8 cm.

Color.—Petal; Light purplish pink (R.H.S. 73C, J.H.S. 8903) with strong reddish purple (R.H.S. 72B, J.H.S. 9208) vein. Bottom color of the corolla throat; moderate reddish purple (R.H.S. 79C, J.H.S. 8913). Outside color of the corolla tube; pale purple (R.H.S. 85D, J.H.S. 8914).

Reproductive organs.—1 normal pistil and 5 normal stamens. Color of pistil is strong yellow green (R.H.S. 144B, J.H.S. 3512). Color of stamen is light olive gray (R.H.S. 197C, J.H.S. 3112).

Peduncle.—1.5 mm in thickness, and 1.5 cm in length.

Physiological and ecological characteristics.—Moderate resistance to cold, heat, diseases and pests. Low resistance to rain.

The pollen parent ‘1792’ used in the crossing of ‘SUNROVE’ was a wild type petunia plant native to Brazil. The petunia plant ‘1792’ has a decumbent habit with long stems and much branching. The leaf is very small without petiole. It has very small single flower, the petals having yellowish white (R.H.S. 158D, J.H.S. 2502) with strong reddish purple (R.H.S. 72B, J.H.S. 9208) vein.

The main botanical characteristics of variety ‘1792’ are as follows:

Plant:

Growth habit.—Decumbent.

Plant height.—5.5 cm.

Spreading area of plant.—Large.

Blooming period.—May to August in the southern Kanto area, Japan.

Stem:

Length.—15.0 cm.

Thickness.—1.3 mm.

Pubescence.—Normal.

Branching.—Much.

Length of internode.—1.0 cm.

Color.—Strong yellow green (R.H.S. 144B, J.H.S. 3512).

Leaf:

Whole shape.—Elliptic. The apex shape is acute, and the base shape is attenuate.

Length.—2.5 cm.

Width.—1.5 cm.

Color.—Upper-side color is strong yellow green (R.H.S. 143A, J.H.S. 3711). Bottom-side color is moderate yellow green (R.H.S. 147B, J.H.S. 3513).

Pubescence.—Sparse.

Flower:

Facing direction.—Horizontal.

Type.—Single.

Shape.—Funnel-shape, with five-fissured limb.

Shape of petal chip.—Round.

Lobation.—Shallow.

Waving of petal.—Weak.

Diameter.—4.3 cm.

Color.—Petal; Yellowish White (R.H.S. 158D, J.H.S. 2502) with strong reddish purple (R.H.S. 72B, J.H.S. 9208) vein. Bottom color of the corolla throat; greenish white (R.H.S. 155C, J.H.S. 3501). Outside color of the corolla tube; brilliant yellow green (R.H.S. 145B, J.H.S. 3504).

Reproductive organs.—1 normal pistil and 5 normal stamens. Color of pistil is strong yellow green (R.H.S. 144B, J.H.S. 3512). Color of stamen is light olive gray (R.H.S. 197C, J.H.S. 3112).

Peduncle.—1.0 mm in thickness, and 2.4 cm in length.

Physiological and ecological characteristics.—Moderate resistance to cold and pests. Strong resistance to rain, heat and diseases.

The main botanical characteristics of a similar variety ‘Revolution Pinkvein’ which was previously filed in Japan [‘Revolution Pinkvein’ The Seed and Seedlings Law Registration No. 4009] and in the United States of America [Petunia Plant ‘Revolution Pinkvein’ (U.S. Plant Pat. No. 9,341)] used for examination as comparison are as follows:

Plant:

Growth habit.—Decumbent. The stems hang down when potted in a hanging pot.

Plant height.—15-25 cm.

Spreading area of plant.—Large.

Blooming period.—April to September in the southern Kanto area, Japan.

Stem:

Length.—60-80 cm.

Thickness.—3.0-4.0 mm.

Pubescence.—Medium.

Branching.—Very abundant (primary), abundant (secondary).

Length of internode.—2.0-4.0 cm.

Color.—Strong yellow green (R.H.S. 144B, J.H.S. 3512).

Leaf:

Leaf attaching angle.—Horizontal.

Whole shape.—Oval. Length — 4.0-5.0 cm.

Width.—2.0-3.5 cm.

Color.—Upper-side color is moderate olive green (R.H.S. 146A, J.H.S. 3508). Bottom-side color is moderate yellow green (R.H.S. 147B, J.H.S. 3513).

Pubescence.—Medium.

Flower:

Facing direction.—Obliquely upward.

Type.—Single.

Shape.—Funnel-shape, with five-fissured limb.

Shape of petal chip.—Round.

Lobation.—Shallow.

Waving of petal.—Weak.

Diameter.—3.5-5.5 cm.

Color.—Petal is bi-color with vein pattern. The ground color is deep purplish pink (R.H.S. 70C, J.H.S. 9213) and pattern color is strong reddish purple (R.H.S. 72A, J.H.S. 9209) — dark reddish purple (R.H.S. 79A, J.H.S. 9218). The bottom throat inside portion of the corolla is a dark reddish purple color (R.H.S. 79A, J.H.S. 9213).

Reproductive organs.—1 normal pistil and 5 normal stamens.

Color of pistil is strong yellow green (R.H.S. 144B, J.H.S. 3512). Color of stamen is light olive gray (R.H.S. 197C, J.H.S. 3112).

Peduncle.—2.0-2.5 mm in thickness, and 0.2-0.3 cm in length.

Physiological and ecological characteristics.—High resistance to cold, rain and heat. Also high resistance to disease. Moderate resistance to pests.

### SUMMARY OF THE VARIETY

The new variety of the petunia plant has a decumbent growth habit, abundant branching and great profusion of blooms with the whole plant remaining in bloom for a considerable period of time. The flowers are single and medium size. The petal color is light purplish pink with vivid reddish purple vein pattern. The plant has a high resistance to rain, heat and diseases.

### BRIEF DESCRIPTION OF THE PHOTOGRAPH

FIG. 1 is a photograph showing a partial view of the new variety of the Petunia plant ‘SUNROVE’ in the ground;

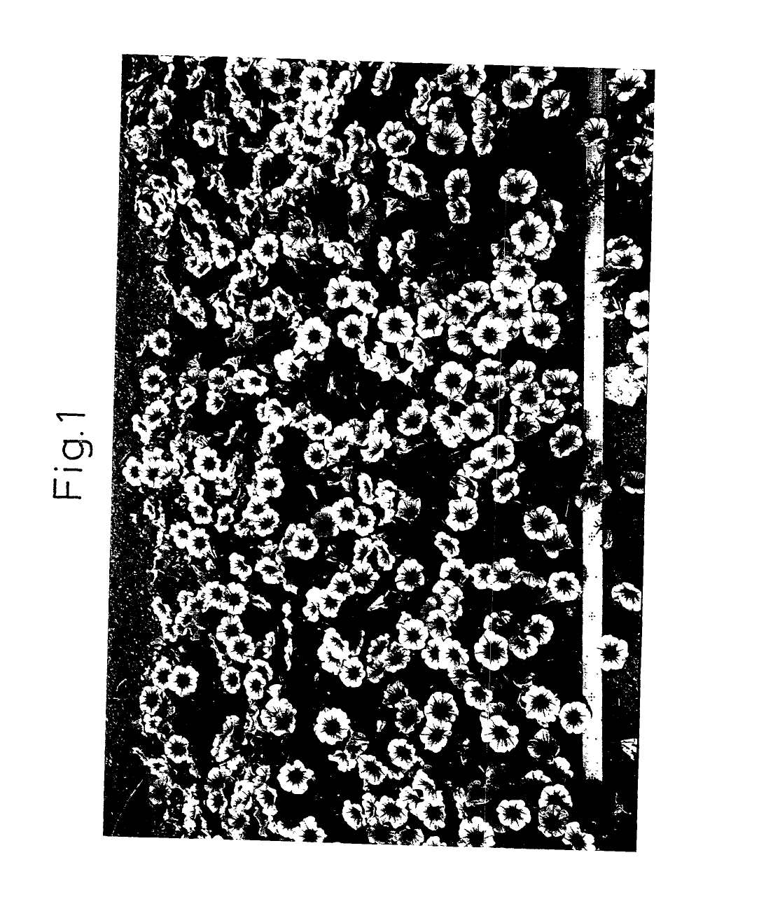

FIG. 2 is a photograph showing a view of the variety of the Petunia plant ‘SUNROVE’ in a flower pot;

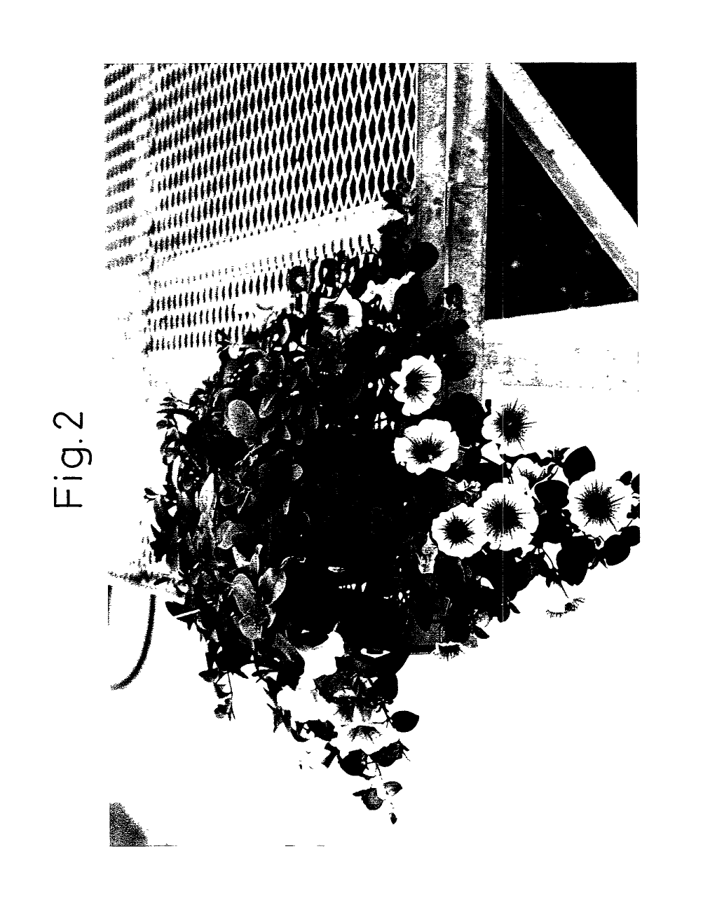

FIG. 3 is a photograph of flowers and leaves of the new variety of the Petunia plant ‘SUNROVE’.

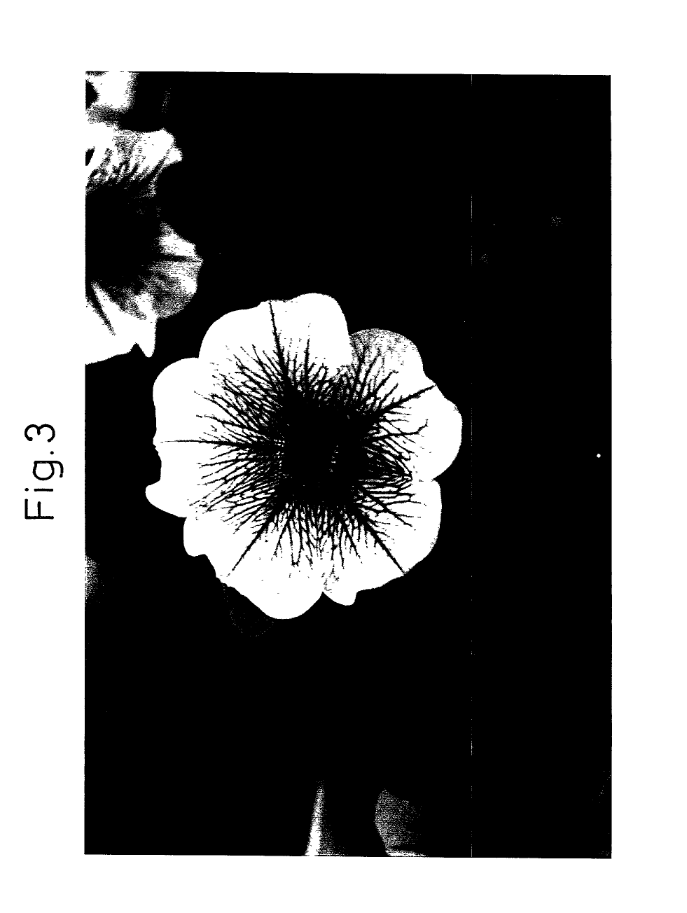

### DESCRIPTION OF THE NEW VARIETY

The botanical characteristics of the new and distinct variety of Petunia plant named ‘SUNROVE’ are as follows.

Plant:

Growth habit.—Decumbent.

Plant height.—10.5 cm.

Spreading area of plant.—Large.

Blooming period.—April to late September in the southern Kanto area, Japan. The plant shape does not change throughout this period. A typical flower commonly lasts approximately 5 days on the plant when experiencing a temperature of approximately 20° C.

Stem:

Length.—12.0 cm.

Thickness.—1.8 mm.

Pubescence.—Dense.

Branching.—Abundant.

Length of internode.—0.9 cm.

Color.—Strong yellow green (R.H.S. 144B, J.H.S. 3512).

Leaf:

Whole shape.—Elliptic. The apex shape is acute, and the base shape is attenuate.

Length.—8.0 cm.

Width.—4.4 cm.

Color.—Upper side color is moderate olive green (R.H.S. 146A, J.H.S. 3508). Bottom side color is moderate yellow green (R.H.S. 147B, J.H.S. 3513).

Thickness.—0.3 mm.

Pubescence.—Medium.

Flower:

Facing direction.—Slanted upward.

Type.—Single.

Shape.—Funnel-shape, with five-fissured limb.

Shape of petal chip.—Round.

Lobation.—Shallow.

Waving of petal.—Weak.

Diameter.—5.5 cm.

Color.—Petal; Light purplish pink (R.H.S. 73C, J.H.S. 8903) with vivid reddish purple (R.H.S. 74A, J.H.S. 9207) vein. Bottom color of the corolla throat; brilliant purple (R.H.S. 81C, J.H.S. 8604). Outside color of the corolla tube; very pale violet (R.H.S. 92D, J.H.S. 8002).

Reproductive organs.—1 normal pistil and 5 normal stamens. Color of pistil is strong yellow green (R.H.S. 144B, J.H.S. 3512). Color of stamen is light purple (R.H.S. 85A, J.H.S. 8304).

Peduncle.—0.7 mm in thickness and 1.7 cm in length.

Physiological and ecological characteristics.—High resistance to rain, heat and diseases. Moderate resistance to cold and pests.

This new variety of Petunia plant is most suitable for flower bedding and potting, particularly in hanging pots or planters, and is excellent for use as ground cover. Pinching of old blossoms will enhance the formation of new blossoms.

I claim:

1. A new and distinct variety of Petunia plant, substantially as herein illustrated and described, characterized particularly as to novelty by (A) having a decumbent habit with long stems, (B) abundant branching and great profusion of blooms, the whole bush remaining in bloom for a considerable period of time, (C) flowers are single and medium size, the petals have a light purplish pink ground color with a vivid reddish purple vein pattern, and the bottom color of the corolla throat is brilliant purple and the outside color of corolla tube is very pale violet, and (D) has a high resistance to rain, heat and diseases.
